(19) United States
(12) Patent Application Publication
(10) Pub. No.: US 2020/0178598 A1
(43) Pub. Date: Jun. 11, 2020

(54) Smart Wireless Water Pipe System For Smoke Sanitization, Storage and Portability

(71) Applicant: Soweto Abijah Mitchell, Hollywood, FL (US)

(72) Inventor: Soweto Abijah Mitchell, Hollywood, FL (US)

(21) Appl. No.: 16/214,071

(22) Filed: Dec. 8, 2018

(51) Int. Cl. A24F 1/30; A24F 47/00; A24F 3/00; A61L 9/03; A61L 9/20; A61L 9/14

(52) U.S. Cl. CPC A24F 1/30; A24F 47/008; A24F 3/00; A61L 2209/11; A61L 9/20; A61L 9/14; A61L 2209/13; A61L 9/03

(57) ABSTRACT

The present invention relates to an electronic water pipe device capable of automatically burning a substance placed into its burner, cooling and to some degree cleansing the resulting smoke of some of its toxins, then, if desired, pressurizing and/or storing all smoke produced in a canister or other gas storage device, for transport or storage. It includes a multicolored light display which responds to music and other audio input. It is also capable of being wirelessly controlled using a PC or Smart Device utilizing a wireless connection through an app or webpage interface/portal, or through voice recognition or on a timer or with input from one or more sensor(s) or the press of one or more buttons be activated and automatically perform to completion all its programmed functions.

### FIELD OF THE INVENTION

The present invention relates to electronic smoking products in general, and more particularly, to a portable electronic water pipe or hookah device.

### BACKGROUND OF THE INVENTION

A water pipe, also known as a Hookah or in some places a Chalice, is a customizably sized, free-standing, air-tight apparatus that traditionally uses air pressure from a smoker sucking on a mouthpiece at the end of a long flexible tube to utilize the force created from the suction to pull air and smoke (produced from burning a substance, e.g. tobacco, located in the “burner” section of the apparatus) through the tube, which is connected to the burner. The combusted air/smoke travels into and then through water or some other cooling medium in the bubbler (“cooler” portion of the apparatus) to filter and cool the smoke. The airtight “Cooler/Bubbler” container housing is filled with a cooling medium, for example water, where smoke is released at the bottom of the water and bubbles up to its surface. It is filtered and cooled by the water, before passing into the user's suction or “exhaust hose” placed through the container wall and suspended above the water level in the same airtight container. A traditional water pipe is typically a single stemmed device with one output whereas a Hookah or Chalice maybe a multi-stemmed, often ceramic, metal or glass-based, instrument for smoking tobacco products as the primary smoking substance. A hookah generally has a bowl, which contains hot coal to ignite the tobacco or desired “shisha” placed in the bowl. A windscreen may be placed over the bowl. A plate is placed under the bowl to receive any falling ash from the coals if coals are utilized. If hot coals aren't utilized then a direct flame or instead an electronically controlled heating element, as used in this invention, may also be employed to ignite the substance.

Once a smoker inhales through the mouthpiece and thus the hose, air is pulled down through the charcoal or flame, where the air is heated, and then the heated air is pulled into the lower portion of the bowl holding the tobacco. The hot air vaporizes the tobacco, thereby producing smoke. The smoke goes down through the “burner tube” into the water. Then, the smoke bubbles up through the water, which receives heat from the smoke and helps filter the smoke. The smoke then fills the top part of the water container. The smoker's suction on the mouthpiece will make the smoke pass through the tube suspended above the water and then into the smoker's mouth. There is a reduction in air pressure in the container when this happens, thus pulling more air through the charcoal, continuing the process.

In this manner, traditional Hookahs are effective but introduce extra toxic elements from the coal and additional heat by not controlling the temperature of the burn and using a toxic fuel to create the burn. A traditional Chalice has no need for coal and uses direct flame to ignite the substance but its design has not become as popular as the Hookah. Advancements in water pipe designs have introduced, electronic vaping-type liquid substances, electronic sensor controls, esp utilizing pressure sensors and electronically controlled heating elements. These are all good enhancements which improve the water pipe/Hookah experience. This invention, however, creates a completely new type of electronic water pipe smoking experience by creating a system which is completely user controllable by means of a WiFi, internet or Bluetooth connection via PC or portable device utilizing and displaying electronic readings of smoke levels and toxicity levels on a users device or through a webserver, for example temperature, carbon monodixe and ammonia levels (and even more data will be monitored in further embodiments) utilizing microcontroller based sensor data processing and electronics control of the device's necessary functions. This device also introduces a UVb and UVc light to photochemically break down certain toxic molecules in the smoke and water while also creating a mesmerizing patterned visual display. The production of additional oxygen through electrolysis is also employed to hopefully provide a healthier experience for the user to ensure minimal reduction of oxygen intake while smoking. Furthermore this device introduces an automatically controlled air pump which allows for high pressure storage of the oxygen/smoke produced by the device in pneumatic canisters or bladders(e.g. balloons) for transportability and storage, also to allow further cooling of the smoke through the expansion, pressurization and depressurization of the gases. In this manner a complete smoke production and storage system is produced where a user is able to place a burning substance, eg tobacco, in the burner, press one button and the system automatically burns it to completion, stores all the smoke produced in the canister or other storage device and turns itself back to standby mode when complete. The same functionality and even more can be accomplished through the web/Wifi/Bluetooth interface. This device can also have one or more additional outlets for traditional Hookah-like usage wherein the user provides the suction. Utilizing this invention would require no physical effort from the user beyond placing the burning substance in the burner and initiating the device. The device has a very sleek and simple form factor with a hourglass-like shape and seemingly only 1 glass tube vertically traversing through the center of the device and an elegant base with 1 or more exhaust filtered smoke/air outlets. The design can vary with one or more than two cooling/bubbling or expansion containers/bottles. This overall design eliminates the need for coal or even direct flames and eliminates most of the smell (since all of the smoke is contained within the device and the smoke/air storage unit) and, through the process of photochemical dissociation, it also removes some of the toxicity associated with burning popular substances like tobacco.

In other embodiments of this invention it may be used as an air filter/cleanser/freshener, as well, where no substance has to be burned but air is pulled from the surrounding environment through the water (or other medium) where it is filtered and cleansed by the water (perhaps infused with essential oils or cleansing chemicals) and to some degree by the UV light present in the water and where the filtered air is then pumped back out into the environment or stored in a canister or bladder for transport, pressurization and/or later consumption. Oxygen and hydrogen are also added into the mix if so desired by the user, or solely oxygen and hydrogen may be generated, taking no air input from the surrounding environment and the newly generated gas output stored in the storage canister/bladder or released into the ambient air. This is accomplished through the process of electrolysis and the production of these gases is microcontroller and sensor data controlled to prevent situations like over-pressurization or overly high concentrations of hydrogen, oxygen or even carbon monoxide levels, etc. If desired by the user, a scented oil or plant/root material could be burned for infusion into the air and scented oils and other essential oils can also be mixed in with the water or replace the water to produce an air freshener or the generated scented air could also be pressurized and stored for later usage. When the device is used as a traditional water pipe/hookah, essential oils or alcohols (without electrolysis) may be introduced into the cooling liquid (water) in order to infuse different flavors and scents into the smoke. The alcohol infusion concept works especially well conceptually with embodiments of this invention that are made with 2 liquor bottles.

### SUMMARY OF THE INVENTION

Certain embodiments of the invention, especially those named as “GO Chalice” or other “Editions” thereof, are based on particular (e.g. customer requested) bottle designs and may be considered portable devices based on the size of the bottle utilized. The general design would include two similar sized bottles or bottle-like containers of the same type where both are aligned vertically (perpendicular to the ground) and the upper bottle (FIG. 3) is placed inverted above the lower bottle (FIG. 1) and connected at the necks by a hollow cylindrical “connector” (FIG. 4) of appropriate diameter. In this way and especially when teardrop or bulb shaped bottles are utilized the water pipe resembles an hour glass shape which is intended (FIG. 5). The bottles/containers, however, don't necessarily have to be bulb or teardrop shaped to achieve this hourglass appearance or for the device to function as intended nor does the device need to be hourglass shaped at all.

In this manner the design is made to be customizable by the intended user or at least feature popular brand bottle shapes which are widely accepted (e.g. famous liquor brands) or other types of containers may be utilized. The neck of the bottles are typically made to be no more than 1″ to 1.5″ from the body of the bottle whereby the connector can be made to be about 1″ to 2″ length and about 1.5″ diameter in order to produce a visually representative hourglass shape. A longer or shorter neck could also be employed as well. The lower bottle is placed on a hollow base which typically is made to be the same shape as the bottom of the lower bottle and that is where all controlling electronics, pump, sensors, and most components of the system are placed. This is done to ensure that the user sees an elegant, transparent, fairly clear of clutter, hourglass-like shape resting on a base with only 2 hardly noticeable transparent tubes running vertically through the bottle necks from the centers of the upper and lower bottles (FIG. 3). The two halves are also easily separable at the bottle neck connector section which facilitates easy cleaning of both halves and easy refilling of the lower cooling bottle. The burner hose detaches out with the upper bottle and the exhaust hose detaches with the lower bottle where it runs into and is connected to the pump within the electronics base unit.

Through an airtight hole from the center of the upper bottle going down through the bottle necks almost to the bottom of the lower bottle is placed the “burner tube” which is typically made of glass or ceramic. Then going up through an airtight hole at the bottom of the lower bottle originating from inside the “electronics housing” section (FIG. 1) and running up through the bottle necks and into the empty space inside the upper bottle is the “exhaust (outlet) hose”. When combusted smoke or normal air travels down from the burner (normally at the top of the device) through the burner tube and into the water in the lower bottle, it then rises up through the water and then up through the bottle necks and then expands into the upper bottle to be sucked back down through the water into the electronics housing section at the base traveling through the exhaust tube. This tube can be transparent, flexible high quality plastic or regular glass/ceramic.

This process allows the smoke to essentially pass through the water once and then briefly again as it passes through the exhaust tube. As the smoke is passing through the water it can be made to mix with additional oxygen produced from the water by 2 or more electrolyzing contacts (which are discretely placed at the bottom of the lower bottle and with wires running through the same watertight hole as the exhaust hose and to which power is provided from the electronics housing and electronically controlled to generate and maintain particular oxygen/hydrogen levels which are sensor detected). The exhaust outlet tube itself may be made conductive and serve as one or both electrodes of the electrolysis circuit (where the outside of the tube could, for example, conduct positive charge where the negative is a separate electrode or concentric hoses could be employed where water is allowed to flow in-between both hoses). The smoke is cooled to some degree while expanding into the upper bottle. This process also allows more time for the UV light to photo-chemically decompose toxins in the smoke and water (FIG. 6). Once the smoke travels down into the electronics housing through the exhaust hose it enters a “sealed air chamber” (FIG. 2) with necessary “sensors” to detect when smoke is present in the system and analyze the contents of the smoke. Once it passes through this air chamber it can be sent through a traditional cartridge type air “filter” and then enters the air pump which pressurizes it and pumps it into the “storage canister” (FIG. 5) which is a new feature of this invention.

The burning substance (e.g. tobacco) may be vaporized by an electronically controlled heating element (in this embodiment a coil of nichrome wire was utilized). The heating element may be attached inside the “burning bowl” or directly to the top of the upper bottle. In this embodiment a separate power source was utilized for the heating element but in another embodiment the burning bowl connects directly at the top of the bottle using transparent conductor lines running up the side of the bottles and both bottles are electrically connected through the use of 2 conductive rings in the middle bottle connector. This heating element is controlled electronically and turns on when the user initiates the device out of standby mode and then turns off when smoke is detected by the sensors indicating that ignition of the substance has occurred. When smoke is no longer detected after first being detected the device shuts itself off with the assumption that the substance is completely burned. Software upgrades to future embodiments could include, for example, doing a redundant burn process after first shut down to ensure all substance is fully consumed and giving the user an option to determine how much oxygenation to add to the process.

In this embodiment the electronics (pump, sensors, heating element, lights, Wifi/Bluetooth/webserver) are controlled by a microcontroller board which is programmed in python and C++ with IoT integration and future possibility for upgradeability. In some embodiments a music player and visual equalizer using UV lights and color LEDs are included as well as additional sensors and charting to keep users up to date with the levels of certain toxins in different substances which will allow them to make more informed consumer choices about which burning substance or brand may be safer. In some embodiments the user is able to produce solely oxygen/hydrogen for storage instead of only smoke and oxygen or solely smoke.

In this embodiment the dimensions of the upper and lower chambers(bottles) were 100 mm diameter base, 20 mm diameter neck and 150 mm height. The base was designed cylindrical to match the bottom of the lower bottle's shape and also has diameter 100 mm with height of 50 mm. The overall height of the device is therefore approx. 350 mm excluding the top “burner” unit. The device is fully scaleable and would only require reducing the size of the base and connector if the overall size was to be reduced if, for instance, smaller dimensioned bottles/containers/flasks were to be utilized. The pump can vary in size to match the size of the upper and lower halves' volume because necessary pumping force may be different (a pump capable of 100 psi or more is desired in order to quickly and effectively charge the storage unit and maintain an even burn). The “burner” intake tube utilized was 240 mm in length, 8 mm in diameter and made of glass to sustain the heat it endures connected to the burner. The exhaust hose was 3 mm pvc air hose of 150 mm length. The base and connector were produced with “glow in the dark” translucent materials which enables the device to glow when being used. Users are encouraged to not touch the burner while the device is still glowing as a reminder. This creates a visually stunning water pipe/hookah otherwise.

### BRIEF DESCRIPTION OF THE DRAWINGS

FIG. 1 is a side view of the lower bottle/container bubbler, exhaust (outlet) hose and connector and electronics base

FIG. 2 is a view of the air sensor exchange unit present in the electronics housing unit and connected to the exhaust (outlet) hose as input and the outlet(canister) hose as output

FIG. 3 is a closeup view of the upper expansion bottle/container and burner tube (burner not shown)

FIG. 5 is a side view of entire device along with the burner on top.

FIG. 6 is a side view of the spinning air outlet which attaches to the burner hose and rotates in the cooling medium when air or smoke flow through the burner tube.

### DETAILED DESCRIPTION

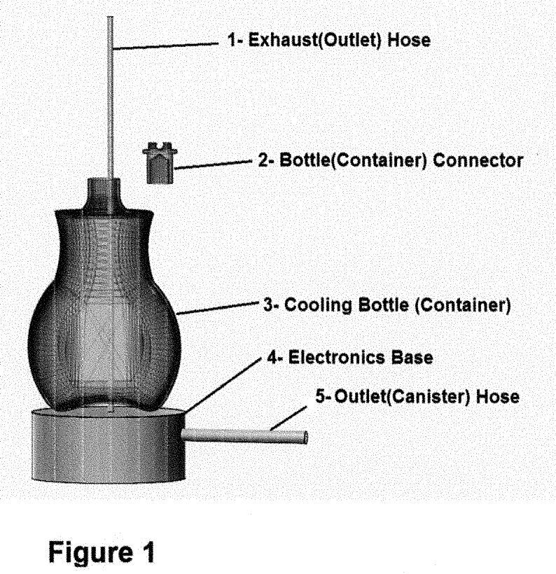

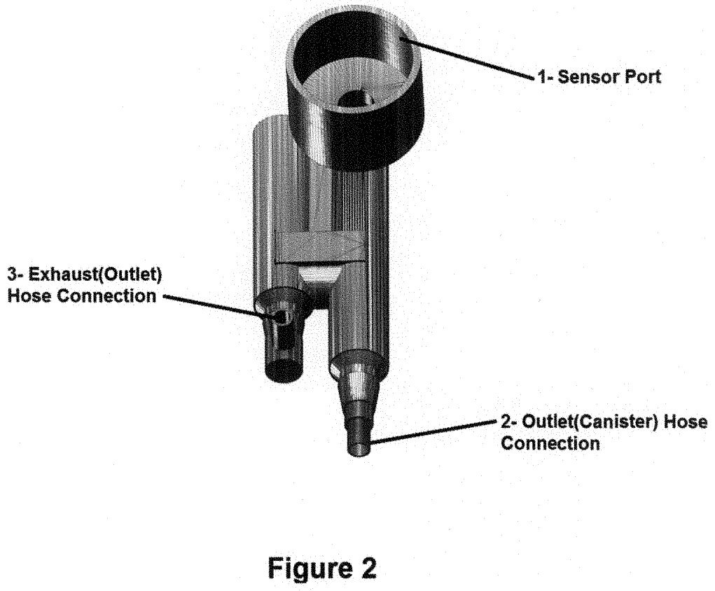

The particular embodiment of the design discussed herein was constructed with dimensions: 38 cm height and 10 cm diameter at the base and top. The preferred middle connector (as shown in FIG. 4) was 4 cm diameter and 5 cm height. The connector was fastened to the lower bottle using transparent epoxy and serves as both connector and handle with or the without the upper bottle adjoined. As shown in FIG. 1 the lower bottle/container was snugly seated on the electronics housing base. A ring of powerful UV leds is placed around the inner circumference of the base's top which cradles the bottom of the lower bottle. These lights are of the type UvB and not only radiate into the bottles but illuminate the outer circumference of the bottles as well. At the center of the base below the lower bottle, with its light projected into the lower bottle and which also shines into the upper bottle, is also placed a small UvC bulb whose ballast is contained within the electronics base along with the mini air pump, air sensor exchange unit and microcontroller unit. In one embodiment a small UV laser is pointed directly up into the Burner tube from the center of the base to better focus the energy. The 1—Exhaust (Outlet) Hose was approx 20 cm in length measured from the base of the lower bottle up to a little higher than the midway point of the upper bottle when both are connected. The hose traverses down the center or along the side of the 3—Cooling Bottle (Container) through a sealed hole in the bottom of the lower bottle and into the 4—Electronics Base where it connects to the input of the air sensor exchange unit shown in FIG. 2. The 5—Outlet (Canister) Hose runs from the pumps output. The pumps tested varied from 3 to 12V DC micro pumps. A 12V unit was selected for this size device. The overall size of the device and components are customizable and can be smaller or larger.

FIG. 2 shows a top-front view of the Air Sensor Exchange Unit where 1 sensor may be situated. Additional Units were constructed which were able to house up to 4 sensors. As shown, 1—Sensor Port is where the sensor input window is placed and sealed off with a thin ring of silicone. Types of sensors utilized all shared same form factor but feature different models for detection of different gases, e.g carbon monoxide, ammonia, hydrogen and combustible gas(smoke). The hose connections labeled 2—Outlet (Canister) Hose Connection and 3—Exhaust (Outlet) Hose Connection are exactly as described.

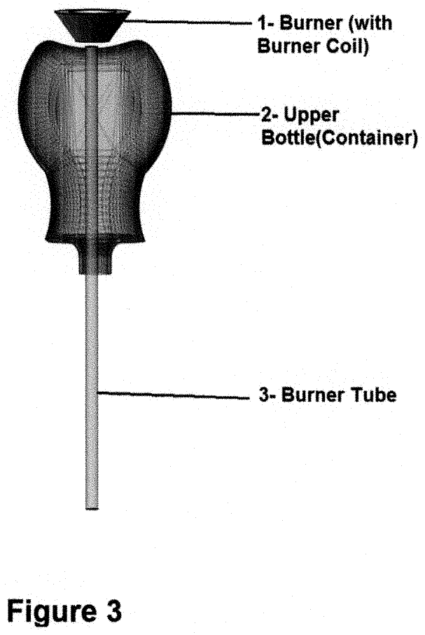

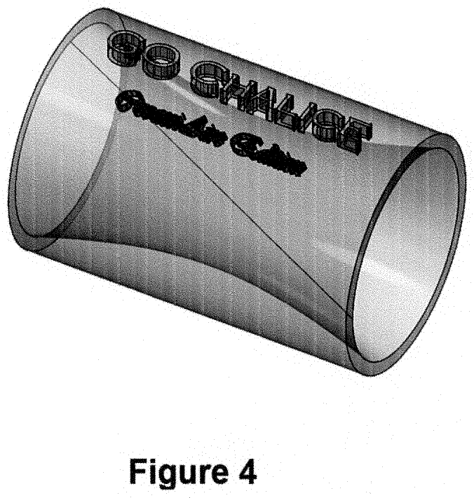

FIG. 3 highlights the components of the upper expansion bottle/container where the smoke which is produced from burning a particular substance in the 1—Burner (with Burner Coil) travels down the 3—Burner Tube where the hot gases are pulled into the cooling medium in the lower bottle and bubble up out of the medium (e.g. water) and rises up through the bottles connector into the 2—Upper Bottle(Container) where the smoke expands and cools before being sucked into the exhaust (outlet) hose shown in FIG. 1. The 2 wires connecting the burner coil aren't shown but are designed to be as thin as possible and placed down the body of the device to be as inconspicuous as possible.

The Bottle Connector shown in FIG. 4 has a wide opening on each end which narrows towards the center of the connector. These gradients within the inner face of the connector are designed to best fit the contours of the bottles or containers being used. In this embodiment the necks of the bottles were shortened to perfectly fit within the connector and appear as close to an hourglass like shape as possible. There are other connector designs and even more may yet be imagined.

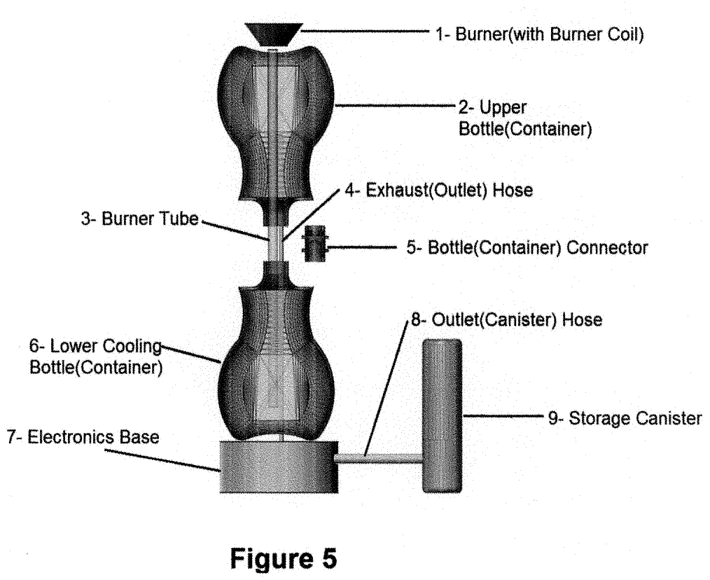

FIG. 5 shows a side view of the entire device from the top 1—Burner to the bottom of the 7—Electronics Base unit. Below the burner running through a sealed hole at the top of the 2—Upper bottle is the 3—Burner tube which is separated from the direct burner heating element by almost 1″ using standard hookah glass adapter fittings and/or a standard hookah burner. The 4—Exhaust (Outlet) Hose runs from the electronics base through the 6—Lower Cooling Bottle(Container) as previously described. The 5—Bottle Connector is not shown connected but it is generally fastened to the mouth of the lower bottle. Shown here is one example of a Bottle Connector whereas FIG. 4 shows another which is slightly different. Once the exhaust smoke/air is pulled from the upper bottle into the Electronics Base unit by the pump, it is then pushed out through the 8—Outlet (Canister) Hose for storage in the 9—Storage canister. A custom canister was designed and constructed using a Schrader Valve as the Input/Output of the Canister. Many things from sealed tubes to balloons were tested and pressure of this embodiment of the system reaches 80 to 100 psi storage pressure with 12V input for the pump using approximately 3 W of power. The canister is designed to be detachable and re-attachable and its size will vary depending on user preferences.

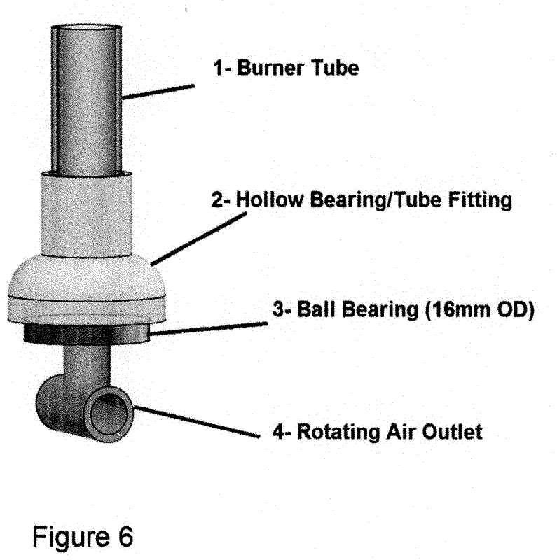

FIG. 6 illustrates another novel innovation in water pipes and hookahs presented by this invention, where a mechanism is shown for allowing smoke/gas/air to freely flow through the burner tube and into the cooling medium while at the same time rotating the outlet(s) at the end of the burner tube in order to cause rotation in the bubbles produced when smoke/gas/air flows through the burner tube. Smoke from the “Burner” travels down through 1—Burner Tube (same “Burner Tube” shown in FIGS. 2 and 5) then through the 2—Hollow Bearing/Tube Fitting and 3—Ball Bearing then out the 4—Rotating Air Outlet which is connected to the Bearing through the axle hole and free to rotate.

1. A water pipe device capable of automatically burning a substance placed into its burner, cooling and to some degree cleansing the resulting smoke of some of its toxins, then, if desired, pressurizing and/or storing all smoke produced in a canister or other gas storage device, for transport or storage.

2. A water pipe device capable of being wirelessly controlled using a PC or Smart Device utilizing a wireless connection through an app or webpage interface/portal, or through voice recognition or on a timer or with input from one or more sensor(s) or the press of one or more buttons be activated and automatically perform to completion all its programmed functions.

3. A water pipe device, as described in claim 1, capable of being used as an air odorizer or freshener when odorizing or scented substances are burned in its burner and which furthermore can store odorized smoke in canisters or other gas storage devices for general storage or atomization and infusion into the ambient air like traditional mist based air fresheners but where the user may refill, store and reuse the canister with whatever scent or gas is created within the system by the user.

4. A water pipe device which filters and cleanses smoke using the process of photochemical decomposition of gases by ultraviolet light.

5. A water pipe device, as described in claim 1, which may be operated like and perform the functions of a Hookah or other recreational smoking device and which may or may not feature a rotating air outlet coupling to cause rotation of bubbles or smoke within the system.

6. A water pipe device, as described in claim 2, which may be operated like and perform the functions of a Hookah or other recreational smoking device and which utilizes different color lights or LEDs that change colors and display patterns in response to inputted audio and which may or may not feature a rotating air outlet coupling to cause rotation of bubbles or smoke within the system

7. A water pipe device, as described in claim 4, which may be operated like and perform the functions of a Hookah or other recreational smoking device and which utilizes different color lights or LEDs that change colors and display patterns in response to inputted audio and which may or may not feature a rotating air outlet coupling to cause rotation of bubbles or smoke within the system

8. An electronic smoking or vaping device, as described in claim 4, which is portable but uses the process of photochemical decomposition by ultraviolet light.

9. A water pipe device, as described in claim 1, capable of producing and storing hydrogen, oxygen or other gases through the process of combustion, electrolysis and/or photochemical decomposition, performed within the system for either infusion into the ambient air, whether mixed with smoke produced or not, or for storage and for which the process can be partially or completely controlled by user input, sensor data, software or a combination thereof.

10. A method, as described in claim 1, for pressurizing and storing the smoke produced from burning a substance in a water pipe, hookah, chalice, vaporizer, odorizer or atomizer device.

11. A method, as described in claim 2, for controlling the functions and general operation of a water pipe, hookah, chalice, vaporizer, odorizer or atomizer device wirelessly and which may also employ sensor data.

12. A method, as described in claim 4, of cleansing or rendering less toxic: smoke, exhaust or fumes using the process of photochemical decomposition by breaking down some or all of the toxic molecules in the smoke into elemental compounds, where the smoke/exhaust/fumes are produced from the combustion of a substance, whether in a water pipe, hookah, chalice, vaporizer, odorizer, atomizer device or any other device which produces smoke including, engines, generators and incinerators.

13. A device, as described in claim 4, capable of cleansing or rendering less toxic: smoke, exhaust or fumes using the process of photochemical decomposition by breaking down some or all of the toxic molecules in the smoke into elemental compounds, where the smoke/exhaust/fumes are produced from the combustion of a substance, whether in a water pipe, hookah, chalice, vaporizer, odorizer, atomizer device or any other device which produces smoke including, engines, generators and incinerators.
